(19) United States
(12) Patent Application Publication
(10) Pub. No.: US 2003/0083385 A1
(43) Pub. Date: May 1, 2003

(54) Use of benzenesulfonyl (thioureas or ure as) for treating of septic shock or gene ralized inflammatory syndrome

(76) Inventors: Uwe Gerlach, Hattersheim (DE); Klaus Wirth, Kriftel (DE); Heinrich Christian Englert, Hofheim (DE); Heinz Gogelein, Frankfurt am Main (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/178,648

(22) Filed: Jun. 24, 2002

(30) Foreign Application Priority Data
Jun. 22, 2001 (DE) 10129704.1

(51) Int. Cl. A61K 31/175; A61K 31/17

(52) U.S. Cl. 514/584; 514/592

(57) ABSTRACT

The present invention is directed to the use of a benzenesulfonyl(thiourea or urea) of formula I

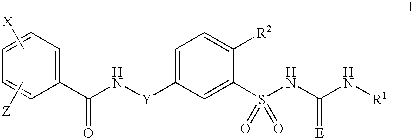

wherein

R¹ is hydrogen, (C₁-C₈)-alkyl-, (C₃-C₈)-cycloalkyl-, (C₃-C₈)-cycloalkyl-(C₁-C₄)-alkyl- or fluoro-(C₁-C₈)-alkyl-;

R² is (C₁-C₆)-alkoxy-, (C₃-C₈)-cycloalkyloxy-, (C₃-C₈)-cycloalkyl-(C₁-C₄)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;

E is oxygen or sulfur;

Y is a hydrocarbon residue of formula —(CR³₂)ₙ—, wherein the residues R³, all independently of each other, are hydrogen or (C₁-C₂)-alkyl, and n is 1, 2, 3 or 4;

X is hydrogen, halogen or (C₁-C₆)-alkyl; and

Z is halogen, (C₁-C₄)-alkyl-, fluoro-(C₁-C₄)-alkyl-, (C₁-C₄)-alkoxy- or fluoro-(C₁-C₄)-alkoxy-, or

a physiologically tolerable salt thereof or solvate thereof, for treating a patient suffering from septic shock or the generalized inflammatory syndrome (SIRS) comprising administering to the patient a pharmaceutically effective amount of the compound of formula I, or a physiologically tolerable salt thereof or solvate thereof.

The invention is also directed to the use of benzenesulfonyl(thioureas or ureas) of formula I, or a physiologically tolerable salt thereof or solvate thereof, for treating a patient suffering from pathological changes in blood pressure due to a septic shock or generalized inflammatory syndrome (SIRS) state, comprising administering to the patient a pharmaceutically effective amount of the compounds of formula I, or a physiologically tolerable salt thereof or solvate thereof.

### FIELD OF THE INVENTION

The present invention relates to the use of benzenesulfonyl(thioureas or ureas) of the formula I

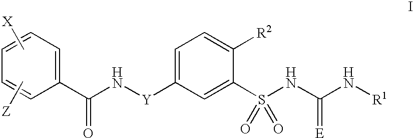

wherein R¹, R², E, X, Y and Z are as defined below, or a physiologically tolerable (pharmaceutically acceptable) salt thereof or solvate thereof, for treating septic shock of a very wide variety of origins and of the generalized inflammatory syndrome, or specifically for treating pathological changes in blood pressure that are associated with the disease patterns of septic shock and the generalized inflammatory syndrome. More specifically, the compounds of formula I are useful for increasing the peripheral (systemic) blood pressure and, at the same time, lower the pulmonary arterial pressure, and thus possess the desired property profile for treating the pathological changes in blood pressure and the cardiovascular problems that are associated with this disease pattern of septic shock and the generalized inflammatory syndrome.

### BACKGROUND OF THE INVENTION

Compounds of formula I are disclosed, for example, in U.S. Pat. No. 5,574,069 (EP-A-612724) and U.S. Pat. No. 5,652,268 (EP-A-727416). These documents report that compounds of formula I selectively inhibit ATP-sensitive potassium channels in the heart and exert a direct antiarrhythmic effect by influencing the duration of the action potential of the heart as a result of the direct effect on the electrical properties of heart muscle cells. Due to this property, the compounds of formula I are suitable, for example, for treating ventricular fibrillation and other cardiac rhythm disturbances. The WO-A-00/15204 document reports that compounds of formula I can also be employed in the treatment and prophylaxis of dysfunctions of the autonomic nervous system. The above documents do not disclose the use of the compounds of formula I for treating septic shock of a very wide variety of origins and of the generalized inflammatory syndrome, or specifically for treating pathological changes in blood pressure that are associated with the disease patterns of septic shock and the generalized inflammatory syndrome.

The disease pattern of sepsis is associated with a general inflammatory reaction and pronounced impairment of hemodynamics, respiration and metabolism which arise, for example, as the result of a massive infiltration of pathogenic bacteria, or their toxins, into the blood circulation. The observation that noxae other than an infection are also able to give rise to very similar disease states led to the introduction of the superordinate concept of the generalized inflammatory syndrome (SIRS, systemic inflammatory response syndrome).

Sepsis and SIRS lead, in particular, to characteristic hemodynamic changes which acutely endanger the blood supply to the body. Sepsis is accompanied by a life-threatening reduction in the systemic blood pressure (generalized circulatory failure; septic shock). Paradoxically, however, the blood pressure (pulmonary arterial pressure) in the lesser circulation, i.e. the pulmonary circulation, can increase in this connection, with this increase possibly constituting a dangerous stress for the right ventricle which further aggravates the overall hemodynamic situation. The right-heart insufficiency that is thereby induced can determine, and dramatically aggravate, the entire cardiovascular situation.

The therapeutic objective when treating the cardiovascular problems that are associated with sepsis or occur in the generalized inflammatory syndrome state would be to at least increase the reduced peripheral blood pressure without (further) increasing the pulmonary arterial pressure. However, it would be ideal if it were possible to lower the pulmonary arterial pressure in addition to increasing the peripheral blood pressure. Vasoconstrictive substances which come into consideration for treating the cardiovascular problems exhibit a favorable effect in the systemic circulation by increasing the peripheral (systemic) blood pressure, however, a simultaneously effected vasoconstriction in the pulmonary vascular system would lead to a (further) increase in the pulmonary arterial pressure and thereby reduce the output from the right ventricle. A pulmonary vasoconstriction can consequently lead to a dangerous reduction in the cardiac minute output and to circulatory collapse.

It would consequently be desirable to have available medicaments which bring about peripheral vasoconstriction without at the same time having a vasoconstrictive effect in the pulmonary vascular system or, even more advantageously, medicaments which even have a vasodilatory effect in the lung. The vasoactive substances which increase both the systemic arterial pressure and the pulmonary arterial pressure, and that have been investigated in animal experiments relating to septic shock or human sepsis, include the benzenesulfonylurea glibenclamide and NO synthase inhibitors (NO=nitric oxide) such as L-NMA (N-methylarginine) or L-NAME (N-nitroarginine methyl ester). However, leaving aside other effects and side-effects, these substances would not, as has been explained, be suitable for treating septic shock because of their hemodynamic effect profile, i.e., the fact that they cause vasoconstriction in both the systemic circulation and in the pulmonary circulation. Further comments in this regard are found in the literature such as, for example, J. Wanstall, Gen. Pharmacol. 1996, 27, 599; M. Dumas et al., Brit. J. Pharmacol. 1997, 120, 405; S. Barman, Am. J. Physiol. 1998, 275, L64; J. Avontuur et al., Crit. Care Med. 1998, 26, 660; R. Weingartner et al., Braz. J. Med. Biol. Res. 1999, 32, 1505; D. Landry et al., J. Clin. Invest. 1992, 89, 2071. It would thus be useful to have compounds that would increase the peripheral (systemic) blood pressure and, at the same time, lower the pulmonary arterial pressure, and consequently possess the desired property profile for treating the pathological changes in blood pressure and the cardiovascular problems that are associated with this disease pattern of septic shock and of the generalized inflammatory syndrome.

### SUMMARY OF THE INVENTION

The present invention is directed to the use of a benzenesulfonyl(thiourea or urea) of formula I

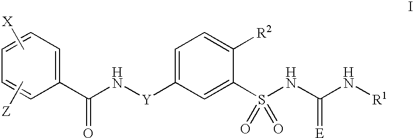

wherein

R¹ is hydrogen, (C₁-C₈)-alkyl-, (C₃-C₈)-cycloalkyl-, (C₃-C₈)-cycloalkyl-(C₁-C₄)-alkyl- or fluoro-(C₁-C₈)-alkyl-;

R² is (C₁-C₆)-alkoxy-, (C₃-C₈)-cycloalkyloxy-, (C₃-C₈)-cycloalkyl-(C₁-C₄)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;

E is oxygen or sulfur;

Y is a hydrocarbon residue of formula —(CR³₂)ₙ—, wherein the residues R³, all independently of each other, are hydrogen or (C₁-C₂)-alkyl, and n is 1, 2, 3 or 4;

X is hydrogen, halogen or (C₁-C₆)-alkyl; and

Z is halogen, (C₁-C₄)-alkyl-, fluoro-(C₁-C₄)-alkyl-, (C₁-C₄)-alkoxy- or fluoro-(C₁-C₄)-alkoxy-, or

a physiologically tolerable salt thereof or solvate thereof, for treating a patient suffering from septic shock or the generalized inflammatory syndrome (SIRS) comprising administering to the patient a pharmaceutically effective amount of the compound of formula I, or a physiologically tolerable salt thereof or solvate thereof; i.e., method of treating a patient suffering from septic shock or the generalized inflammatory syndrome (SIRS) comprising administering to the patient a pharmaceutically effective amount of the compound of formula I, or a physiologically tolerable salt thereof or solvate thereof.

The invention is also directed to the use of benzenesulfonyl(thioureas or ureas) of formula I, or a physiologically tolerable salt thereof or solvate thereof, for treating a patient suffering from pathological changes in blood pressure due to a septic shock or generalized inflammatory syndrome (SIRS) state, comprising administering to the patient a pharmaceutically effective amount of the compounds of formula I, or a physiologically tolerable salt thereof or solvate thereof, i.e., method of treating a patient suffering from pathological changes in blood pressure due to a septic shock or the generalized inflammatory syndrome (SIRS) comprising administering to the patient a pharmaceutically effective amount of the compound of formula I, or a physiologically tolerable salt thereof or solvate thereof.

The invention is also directed to the use of benzenesulfonyl(thioureas or ureas) of formula I, or a physiologically tolerable salt thereof or solvate thereof, for treating a patient suffering a decrease in peripheral (systemic) blood pressure and, at the same time, an increase in pulmonary arterial pressure, comprising administering to the patient a pharmaceutically effective amount of the compounds of formula I, or a physiologically tolerable salt thereof or solvate thereof, i.e., method of treating a patient suffering from decrease in peripheral (systemic) blood pressure and, at the same time, increase in pulmonary arterial pressure, comprising administering to the patient a pharmaceutically effective amount of the compound of formula I, or a physiologically tolerable salt thereof or solvate thereof.

### DETAILED DESCRIPTION OF THE INVENTION

Definitions of Terms

As used above, and throughout the description of the invention, the following terms, unless otherwise indicated, shall be understood to have the following meanings.

The term “treating pathological changes in blood pressure” also encompasses preventing or obviating or alleviating pathological changes in blood pressure due to the septic shock or generalized inflammatory syndrome state.

Alkyl is straight-chain or branched saturated hydrocarbon residues. This also applies when the alkyl residue is substituted, as in fluoroalkyl residues for example, or occurs as a substituent on another residue, for example in alkoxy residues or fluoroalkoxy residues. Examples of straight-chain and branched alkyl residues are methyl, ethyl, n-propyl, isopropyl, n-butyl, sec-butyl, isobutyl, tert-butyl, n-pentyl, isopentyl, neopentyl, n-hexyl, isohexyl, n-heptyl and n-octyl.

Examples of cycloalkyl residues are cyclopropyl, cyclobutyl, cyclopentyl, cyclohexyl, cycloheptyl or cyclooctyl. Cycloalkyl residues can additionally carry one or more, for example, 1, 2, 3 or 4, identical or different (C₁-C₄)-alkyl residues or (C₁-C₄)-fluoroalkyl residues, for example methyl groups or trifluoromethyl groups. Examples of cycloalkyl-alkyl-residues are cyclopropylmethyl-, cyclobutylmethyl-, cyclopentylmethyl-, cyclohexylmethyl-, cycloheptylmethyl-, cyclooctylmethyl-, 1-cyclopropylethyl-, 2-cyclopropylethyl-, 1-cyclopentylethyl-, 2-cyclopentylethyl-, 1-cyclohexylethyl-, 2-cyclohexylethyl-, 3-cyclopropylpropyl-, 3-cyclopentylpropyl-, 3-cyclohexylpropyl- and 4-cyclopropylbutyl-.

Examples of the alkoxy (=alkyloxy) residue that is bonded via an oxygen atom are methoxy, ethoxy, n-propoxy, isopropoxy, n-butoxy, isobutoxy, tert-butoxy, n-pentoxy, neopentoxy and isohexoxy. Examples of the cycloalkyloxy residue are cyclopropyloxy, cyclobutyloxy, cyclopentyloxy and cyclohexyloxy.

Fluoroalkyl is an alkyl residue wherein one or more hydrogen atoms of an alkyl residue, that is defined as above, have been replaced with fluorine atoms. One or more fluorine atoms, for example 1, 2, 3, 4, 5, 6 or 7, can be present in a fluoroalkyl residue. As a maximum, all the hydrogen atoms can be replaced, that is perfluorosubstitution can be present. Examples of fluoroalkyl are fluoromethyl, difluoromethyl, trifluoromethyl, 2,2,2-trifluoroethyl and pentafluoroethyl. Fluoroalkoxy is an alkoxy residue that is defined as above and wherein, as explained, one or more hydrogen atoms, for example one, two, three or four hydrogen atoms, have been replaced with fluorine atoms. Examples of fluoroalkoxy are trifluoromethoxy and 2,2,2-trifluoroethoxy.

The above definitions and explanations also apply, independently of each other, to all alkyl groups in the alkoxy-alkoxy- and alkoxy-alkoxy-alkoxy-residues which residues are bonded via an oxygen atom. In the divalent alkyl groups that are contained in these groups, the two free bonds by which these groups are bonded to the neighboring groups can be present in any positions, for example in the 1,1 position of an alkyl residue, in the 1,2 position, in the 1,3 position or in the 1,4 position. Examples of such divalent residues are methylene, 1,2-ethylene, 1,2-propylene, 1,3-propylene, 1,4-butylene and 2,2-dimethyl-1,3-propylene. A preferred divalent residue of this nature is 1,2-ethylene. Examples of alkoxy-alkoxy-residues are methoxy-methoxy-, 2-methoxy-ethoxy-, 3-methoxy-propoxy-, 4-methoxy-butoxy-, 6-methoxy-hexoxy-, 2-ethoxy-ethoxy-, 2-ethoxy-2-methyl-ethoxy-, 3-ethoxy-propoxy-, 2-propoxy-ethoxy-, 2-isobutoxy-ethoxy- and 2-tert-butoxy-ethoxy-. Examples of alkoxy-alkoxy-alkoxy-residues are (2-methoxy-ethoxy)-methoxy-, 2-(2-methoxy-ethoxy)-ethoxy-, 2-(2-isopropoxy-ethoxy)-ethoxy-, 2-(2-n-butoxy-ethoxy)-ethoxy-, 3-(2-methoxy-ethoxy)-propoxy- and 2-(2-methoxy-2-methyl-ethoxy)-2-methyl-ethoxy-.

Examples of halogen are fluorine, chlorine, bromine and iodine, in particular fluorine and chlorine.

Compounds of formula I for use according to the present invention encompasses any stereoisomeric form thereof or mixtures thereof in all ratios. Centers of asymmetry that are present in the compounds of formula I, for example in the Y group or in alkyl groups, can all, independently of each other, exhibit the S configuration or the R configuration. All possible enantiomers and diastereomers, as well as mixtures of two or more stereoisomeric forms, for example mixtures of enantiomers and/or diastereomers, in all ratios, are comprised by the invention. Thus, enantiomers are a subject of the invention in enantiomerically pure form, both as levorotatory and as dextrorotatory antipodes, in the form of racemates and in the form of mixtures of the two enantiomers in all ratios. Diastereomers are a subject-matter of the invention both in pure form and in the form of mixtures of two or more diastereomers in all ratios. The invention also encompasses meso compounds. When a cis/trans isomerism is present, both the cis form and the trans form and mixtures of these forms in all ratios are a subject of the invention. If desired, individual stereoisomers can be prepared by fractionating a mixture using customary methods, for example chromatography or crystallization, or by using stereochemically homogeneous starting substances in the synthesis. Where appropriate, a derivatization can be carried out before stereoisomers are separated. A stereoisomeric mixture can be separated at the level of the compounds of formula I or at the level of an intermediate during the course of the synthesis. The invention also encompasses all tautomeric forms of the compounds of formula I.

Physiologically tolerated salts of the compounds of formula I are, in particular, pharmaceutically utilizable salts or nontoxic salts. They can contain inorganic or organic salt components (see Remington's Pharmaceutical Sciences, A. R. Gennaro (Editor), Mack Publishing Co., Easton Pa., 17^{th} edition, 1985, page 1418). These salts can be prepared, for example, from compounds of formula I using suitable inorganic or organic bases, for example using basic alkali metal or alkaline earth metal compounds such as sodium hydroxide or potassium hydroxide, or using ammonia or organic amino compounds or ammonium hydroxides. In general, reactions of compounds of formula I with bases for the purpose of preparing the salts are carried out in accordance with customary procedures in a solvent or diluent, for example in an alcohol such as methanol. Because of their physiological and chemical stability, advantageous salts are in many cases sodium, potassium, magnesium or calcium salts or ammonium salts, in particular sodium salts. Formation of a salt on the sulfonyl group-substituted nitrogen atom of the urea or thiourea group leads to compounds of formula II

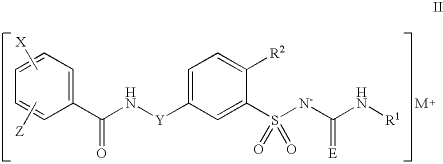

wherein R¹, R², E, X, Y and Z are as defined above and M is a cation M, for example, an alkali metal ion or one equivalent of an alkaline earth metal ion, for example the sodium, potassium, magnesium or calcium ion, or is the unsubstituted ammonium ion or an ammonium ion having one or more organic residues. An ammonium ion standing for M can, for example, also be the cation that is obtained, by protonation, from an amino acid, in particular a basic amino acid such as lysine or arginine.

The present invention also encompasses solvates of compounds of formula I and their physiologically tolerated salts, for example hydrates or adducts with alcohols, and also derivatives of the compounds of formula I and prodrugs and active metabolites.

Particular or Preferred Embodiments

In formula I, R¹ is preferably hydrogen, (C₁-C₈)-alkyl-, (C₃-C₈)-cycloalkyl- or (C₁-C₈)-fluoroalkyl-; particularly preferably hydrogen or (C₁-C₆)-alkyl-, very particularly preferably hydrogen or (C₁-C₄)-alkyl-, especially preferably (C₁-C₄)-alkyl-, in particular methyl.

If R² is (C₁-C₆)-alkoxy- in formula I, the residue is then preferably (C₁-C₄)-alkoxy-, in particular methoxy or ethoxy, especially methoxy. If R² is (C₁-C₆)-alkoxy-(C₁-C₄)-alkoxy- in formula I, the residue is then preferably (C₁-C₄)-alkoxy-(C₁-C₄)-alkoxy-, in particular 2-((C₁-C₄)-alkoxy)-ethoxy-, especially 2-methoxy-ethoxy-. If R² is (C₁-C₆)-alkoxy-(C₁-C₄)-alkoxy-(C₁-C₄)-alkoxy- in formula I, the residue is then preferably (C₁-C₄)-alkoxy-(C₁-C₄)-alkoxy-(C₁-C₄)-alkoxy-, in particular 2-(2-((C₁-C₄)-alkoxy)-ethoxy)-ethoxy-, especially 2-(2-methoxy-ethoxy)-ethoxy-. A group of preferred residues R² is formed by the residues (C₁-C₆)-alkoxy, (C₁-C₆)-alkoxy-(C₁-C₄)-alkoxy- and (C₁-C₆)-alkoxy-(C₁-C₄)-alkoxy-(C₁-C₄)-alkoxy-, in particular the residues (C₁-C₆)-alkoxy and (C₁-C₆)-alkoxy-(C₁-C₄)-alkoxy-, especially the residues methoxy and 2-methoxy-ethoxy-, very especially the residue 2-methoxy-ethoxy-.

The residues R³ are preferably, independently of each other, hydrogen or methyl, particularly preferably hydrogen.

n is preferably 2 or 3, particularly preferably 2.

The group Y preferably contains up to four carbon atoms. Particularly preferably, Y is the group —(CH₂)ₙ— wherein n is 2 or 3, or is the group —CHR³—CH₂— wherein R³ is methyl or ethyl and the group —CHR³— is bonded to the NH group. Very particularly preferably, Y is the group —CH₂—CH₂—.

X is preferably hydrogen, halogen or (C₁-C₄)-alkyl-, particularly preferably halogen, for example fluorine, chlorine, methyl, ethyl, n-propyl, isopropyl, n-butyl, isobutyl, sec-butyl or tert-butyl, in particular fluorine or chlorine, especially chlorine. Z is preferably halogen, (C₁-C₄)-alkoxy- or (C₁-C₄)-alkyl-, particularly preferably (C₁-C₄)-alkoxy-, for example methoxy or ethoxy, especially methoxy. The residues X and Z can be located in all positions of the phenyl residue to which they are bonded. Preferably, X is bonded in the 5 position and Z in the 2 position of the phenyl residue, in each case with reference to the group C(═O)—NH in the 1 position.

If the group E in the compounds of formula I is oxygen, then the compounds are ureas of formula Ia. If the group E in the compounds of formula I is sulfur, then the compounds are thioureas of formula Ib.

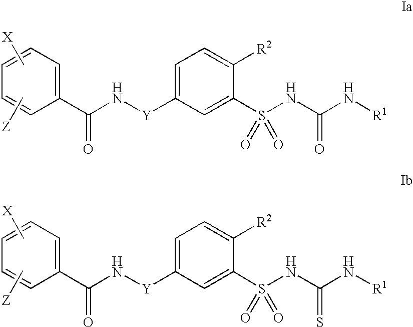

Compounds of formula I that are preferred for the use according to the invention are compounds wherein one or more of the residues have preferred or particular meanings, with all combinations of preferred or particular meanings being a subject of the present invention.

Thus, for example, preference is given to using a compound of formula I

wherein

R¹ is hydrogen, (C₁-C₈)-alkyl-, (C₃-C₈)-cycloalkyl or fluoro-(C₁-C₈)-alkyl-;

R² is (C₁-C₆)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;

E is oxygen or sulfur;

Y is a hydrocarbon residue of formula —(CR³₂)ₙ—, wherein the residues R³, all independently of each other, are hydrogen or (C₁-C₂)-alkyl-, and n is 1, 2, 3 or 4;

X is hydrogen, halogen or (C₁-C₄)-alkyl-; and

Z is halogen, (C₁-C₄)-alkyl- or (C₁-C₄)-alkoxy-, or

a physiologically tolerable salt thereof or solvate thereof.

Particular preference is given to using a compound of formula I wherein

R¹ is hydrogen or (C₁-C₆)-alkyl-;

R² is (C₁-C₆)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;

E is oxygen or sulfur;

Y is a hydrocarbon residue of formula —(CR³₂)ₙ—, wherein the residues R³, all independently of each other, are hydrogen or (C₁-C₂)-alkyl-, and n is 1, 2, 3 or 4;

X is hydrogen, halogen or (C₁-C₄)-alkyl-; and

Z is halogen, (C₁-C₄)-alkyl- or (C₁-C₄)-alkoxy-, or

a physiologically tolerable salt thereof or solvate thereof.

A very particularly preference is given to using a compound of formula I

wherein

R¹ is hydrogen or (C₁-C₆)-alkyl-;

R² is methoxy or 2-methoxy-ethoxy-;

E is oxygen or sulfur;

Y is a hydrocarbon residue of formula —(CR³₂)ₙ—, wherein the residues R³, all independently of each other, are hydrogen or methyl, and n is 2 or 3;

X is hydrogen, halogen or (C₁-C₃)-alkyl-; and

Z is halogen, (C₁-C₃)-alkyl or (C₁-C₃)-alkoxy, or

a physiologically tolerable salt thereof or solvate thereof.

Special preference is given to using compounds of formula I wherein

R¹ is (C₁-C₄)-alkyl-;

R² is methoxy or 2-methoxy-ethoxy-;

E is oxygen or sulfur;

Y is the hydrocarbon residue of formula —(CR³₂)ₙ—, wherein the residues R³ all are hydrogen, and n is 2;

X is chlorine, fluorine or (C₁-C₃)-alkyl-;

Z is chlorine, fluorine, (C₁-C₃)-alkyl- or (C₁-C₃)-alkoxy-, or

a physiologically tolerable salt thereof or solvate thereof.

Additionally, preference is given, on the one hand, to using a compound of formula I wherein

R¹ is methyl;

R² is methoxy;

E is sulfur;

Y is the divalent residue —CH₂—CH₂—;

X is chlorine; and

Z is methoxy, or

a physiologically tolerable salt thereof or solvate thereof,

or to using a compound of formula I wherein

R¹ is methyl;

R² is 2-methoxy-ethoxy-;

E is sulfur;

Y is the divalent residue —CH₂—CH₂—;

X is chlorine; and

Z is methoxy, or

a physiologically tolerable salt thereof or solvate thereof.

Examples of compounds of formula I that can be used according to the invention are 1-(5-(2-(5-chloro-2-methoxybenzamido)ethyl)-2-(2-methoxyethoxy)-phenylsulfonyl)-3-methylthiourea, or a physiologically tolerable salt thereof or solvate thereof, for example the sodium salt, and 1-(5-(2-(5-chloro-2-methoxybenzamido)-ethyl)-2-methoxy-phenylsulfonyl)-3-methylthiourea or a physiologically tolerable salt thereof or solvate thereof, for example the sodium salt. These two compounds can also be designated, for example, as 5-chloro-2-methoxy-N-(2-(3-methylaminothio-carbonylaminosulfonyl-4-(2-methoxyethoxy)-phenyl)ethyl)benzamide and 5-chloro-2-methoxy-N-(2-(3-methylaminothiocarbonylaminosulfonyl-4-methoxyphenyl)ethyl)-benzamide.

The compounds of formula I can be prepared, for example, by means of the following processes.

(a) Aromatic sulfonamides of formula III, or their salts of formula IV, can be reacted with R¹-substituted isocyanates of formula V to give substituted benzenesulfonylureas of formula Ia.

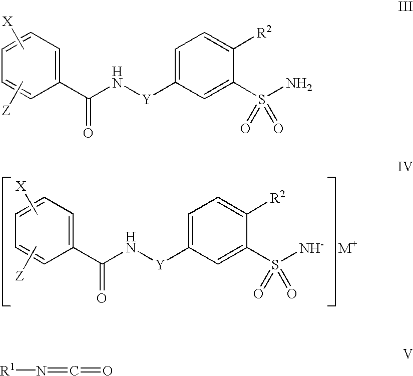

M¹ is a cation that is suitable for use as part of a salt of formula IV and is an alkali metal ion or alkaline earth metal ions such as sodium ion or potassium ion, or an ammonium ion such as, for example, tetraalkylammonium ion. Instead of the R¹-substituted isocyanates of formula V, also R¹-substituted carbamic acid esters, R¹-substituted carbamoyl halides or R¹-substituted ureas can be used in an equivalent manner.

(b) Benzenesulfonylureas of formula Ia that are unsubstituted at the terminal nitrogen atom of the urea group and wherein R¹ is hydrogen, can be prepared by reacting aromatic benzenesulfonamides of formula III, or their salts of formula IV, with trialkylsilyl isocyanates, such as trimethylsilyl isocyanate, or with silicon tetraisocyanate, and hydrolyzing the silicon-substituted benzenesulfonylureas that are initially formed. Furthermore, compounds of formula Ia wherein R¹ is hydrogen can be obtained from benzenesulfonamides of formula III, or their salts of formula IV, by reacting them with cyanogen halides and hydrolyzing the N-cyanosulfonamides, that are formed initially, with mineral acids at temperatures of from about 0° C. to about 100° C.

(c) Benzenesulfonylureas of formula Ia can be prepared from aromatic benzenesulfonamides of formula III, or their salts of formula IV, and R¹-substituted trichloroacetamides of formula VI in the presence of a base in an inert solvent, in accordance with Synthesis 1987, 734, at temperatures of from about 25° C. to about 150° C.

Cl₃C—CO—NH—R¹ VI

Examples of suitable bases are alkali metal or alkaline earth metal hydroxides, hydrides, amides or alcoholates, such as sodium hydroxide, potassium hydroxide, calcium hydroxide, sodium hydride, potassium hydride, calcium hydride, sodium amide, potassium amide, sodium methoxide, sodium ethoxide, potassium methoxide or potassium ethoxide. Suitable inert solvents are ethers, such as tetrahydrofuran, dioxane or ethylene glycol dimethyl ether (DME), ketones, such as acetone or butanone, nitriles, such as acetonitrile, nitro compounds, such as nitromethane, esters, such as ethyl acetate, amides, such as dimethylformamide (DMF) or N-methylpyrrolidone (NMP), hexamethylphosphoric triamide, sulfoxides such as dimethyl sulfoxide (DMSO), sulfones, such as sulfolane, and hydrocarbons, such as benzene, toluene and xylenes. Mixtures of these solvents with one another are also suitable.

(d) Benzenesulfonylthioureas of formula Ib can be prepared from benzenesulfonamides of formula III, or their salts of formula IV, and R¹-substituted isothiocyanates of formula VII.

R¹—N═C═S VII

(e) Benzenesulfonylthioureas of formula Ib that are unsubstituted at the terminal nitrogen atom of the thiourea group and wherein R¹ is hydrogen, can be prepared by reacting aromatic benzenesulfonamides of formula III, or their salts of formula IV, with trialkylsilyl isothiocyanates, such as trimethylsilyl isothiocyanate, or with silicon tetraisothiocyanate, and hydrolyzing the silicon-substituted benzenesulfonylthioureas that are formed initially. It is furthermore possible, in order to prepare compounds of formula Ib wherein R¹ is hydrogen, to react aromatic benzenesulfonamides of formula III, or their salts of formula IV, with benzoyl isothiocyanate and then react the intermediary benzoyl-substituted benzenesulfonylthioureas with aqueous mineral acids. Similar processes are described in J. Med. Chem. 1992, 35, 1137.

(f) Substituted benzenesulfonylureas of formula Ia can be prepared from benzenesulfonylthioureas of formula Ib by means of transformation reactions. The preparation of a benzenesulfonylurea of formula Ia by desulfurization, i.e. the replacement of the sulfur atom in the thiourea moiety of the respective benzenesulfonylthiourea with an oxygen atom, can be performed, for example, with the aid of oxides or salts of heavy metals or by using oxidizing agents such as hydrogen peroxide, sodium peroxide or nitrous acid. Benzenesulfonylthioureas can also be desulfurized by treating them with phosgene or phosphorus pentachloride. As intermediates chloroformic amidines or carbodiimides are obtained that can be converted into the corresponding substituted benzenesulfonylureas by hydrolysis or the addition of water, for example.

(g) Benzenesulfonylureas of formula Ia can be prepared from benzenesulfonyl halides of formula VIII using R¹-substituted ureas or R¹-substituted bis(trialkylsilyl)ureas. Standard methods can be used to remove the trialkylsilyl protecting group from the primarily resulting (trialkylsilyl)benzenesulfonylureas. Furthermore, the sulfonyl chlorides of formula VIII can be reacted with parabanic acids to give benzenesulfonylparabanic acids, whose hydrolysis with mineral acids yields the corresponding benzenesulfonylureas of formula Ia.

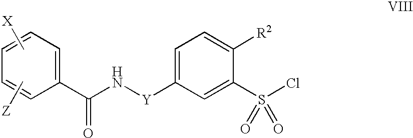

(h) Benzenesulfonylureas of formula Ia can be prepared by reacting amines of formula R¹—NH₂ with benzenesulfonyl isocyanates of formula IX. In the same way, amines of formula R¹—NH₂ can be reacted with benzenesulfonylcarbamic esters, with benzenesulfonylcarbamoyl halides or with benzenesulfonylureas of formula Ia wherein R¹ is hydrogen, to give compounds of formula Ia.

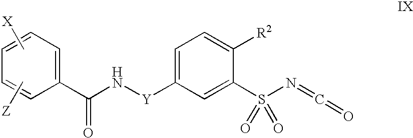

(i) Benzenesulfonylthioureas of formula Ib can be prepared by reacting amines of formula R¹—NH₂ with benzenesulfonyl isothiocyanates of formula X. In the same way, amines of formula R¹—NH₂ can be reacted with benzenesulfonylcarbamic thioesters or benzenesulfonylcarbamoyl thiohalides to give compounds of formula Ib.

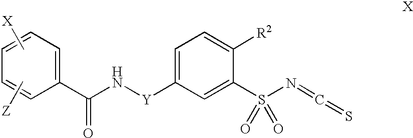

(k) Correspondingly substituted benzenesulfenylureas or benzenesulfinylureas can be oxidized to give benzenesulfonylureas of formula Ia using oxidizing agents such as hydrogen peroxide, sodium peroxide or nitrous acid.

The starting compounds for the abovementioned processes for synthesizing the compounds of formula I can be prepared using methods that are known per se and are described in the literature (for example in the standard works such as Houben-Weyl, Methoden der Organischen Chemie [Methods of Organic Chemistry], Georg Thieme Verlag, Stuttgart; Organic Reactions, John Wiley & Sons, Inc., New York; or in the abovementioned patent specifications), under reaction conditions that are known and suitable for said reactions. It is also possible to make use of variants that are known per se but that are not mentioned here in detail. If desired, the starting compounds can also be formed in situ such that they are not isolated from the reaction mixture but are immediately subjected to further reaction.

Suitably substituted amines of formula XI can be acylated and subjected to a halosulfonation. R² and Y in formula XI have the meanings mentioned above with respect to formula I, however, in addition R² in formula XI can also be a precursor of one of the abovementioned groups, which precursor is then converted, in one or more subsequent steps, into the final R² group. Suitable acylating agents R⁴—COB for acylating the amino group in the compounds of formula XI are alkyl esters, halides (for example chlorides or bromides) or anhydrides of carboxylic acids.

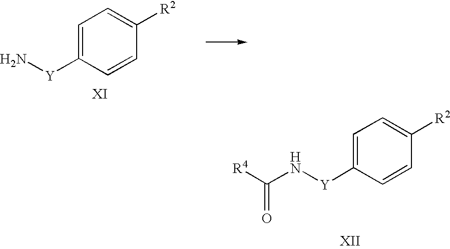

R⁴ is, for example, a trihalomethyl residue, a (C₁-C₄)-alkyl residue or a phenyl residue. If R⁴ is a phenyl residue, the compound of formula R⁴—COB is a benzoic acid derivative. The benzoic acid derivative can be unsubstituted or substituted, for example by one or two identical or different residues such as X and Z, with X and Z being defined as above with respect to formula I. Thus, X can be hydrogen, (C₁-C₆)-alkyl or halogen, and Z can be halogen, (C₁-C₄)-alkyl, fluoro-(C₁-C₄)-alkyl-, (C₁-C₄)-alkoxy or fluoro-(C₁-C₄)-alkoxy-. The group B is a leaving group, such as halogen, (C₁-C₄)-alkoxy, trihaloacetoxy or (C₁-C₄)-alkylcarbonyloxy, for example. Examples of compounds of formula R⁴—COB are acetic anhydride, trihaloacetic anhydride, such as trifluoracetic anhydride, acetyl halides, trihaloacetyl halides, propionyl chloride, isobutyryl bromide, isobutyryl chloride, formic/acetic anhydride, benzoyl chloride and substituted benzoic acid derivatives such as 5-chloro-2-methoxybenzoyl chloride, 5-chloro-2-methoxybenzoic anhydride, (C₁-C₄)-alkyl 5-chloro-2-methoxybenzoate, 5-tert-butyl-2-methoxybenzoyl chloride or 2,5-difluorobenzoyl chloride. The syntheses of the compound of formula XII are preferably carried out in the presence of a tertiary amine base, such as pyridine or a trialkylamine, in the presence or absence of an inert solvent, it also being possible for a catalyst such as dimethylaminopyridine to be present. In general, the reaction is carried out at temperatures of from about 0° C. to about 160° C., preferably from about 20° C. to about 150° C. The acyl group in the compound of formula XII can be either a protecting group or, in the case of the benzoic acid derivatives, a part of the final compound of formula I. Examples of suitable inert solvents for the acylation are ethers, such as tetrahydrofuran, dioxane, or glycol ethers, such as ethylene glycol monomethyl ether or ethylene glycol monoethyl ether (methyl glycol or ethyl glycol) or ethylene glycol dimethyl ether, ketones, such as acetone or butanone, nitriles, such as acetonitrile, nitro compounds, such as nitromethane, esters, such as ethyl acetate, amides, such as DMF or NMP, hexamethylphosphoric triamide, sulfoxides, such as DMSO, chlorinated hydrocarbons, such as dichloromethane, chloroform, trichloroethylene, 1,2-dichloroethane or carbon tetrachloride, or hydrocarbons, such as benzene, toluene or xylenes. Mixtures of these solvents with one another are also suitable.

The sulfonamides of formula XIII can be prepared from the compounds of formula XII using methods that are known per se, employing reaction conditions

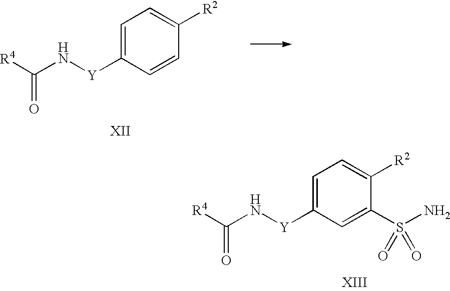

that are known and suitable for such reactions. It is also possible to make use of variants that are known per se but that are not mentioned here in detail. If desired, the syntheses can be carried out in one, two or more steps. In particular, preference is given to processes wherein the acylated amine of formula XII is converted, using electrophilic reagents, in the presence or absence of inert solvents at temperatures of from about −10° C. to about 120° C., preferably from 0° C. to about 100° C., into aromatic sulfonic acids or their derivatives, such as sulfonyl halides. For example, it is possible to carry out sulfonations using sulfuric acids or fuming sulfuric acid, halosulfonations using halosulfonic acids, reactions with sulfuryl halides in the presence of anhydrous metal halides, or reactions with thionyl halides in the presence of anhydrous metal halides with a subsequent oxidation carried out in a known manner to give aromatic sulfonyl chlorides. If sulfonic acids are the primary reaction products, these can then be either converted directly, or after treatment with tertiary amines, such as pyridine or trialkylamines, or with alkali metal or alkaline earth metal hydroxides or reagents which form these basic compounds in situ, in a known manner into sulfonyl halides, using acid halides such as phosphorus trihalides, phosphorus pentahalides, phosphorus oxychlorides, thionyl halides or oxalyl halides. The sulfonic acid derivatives can be converted into sulfonamides in a manner known from the literature. Preference is given to reacting the sulfonyl chlorides, in an inert solvent and at temperatures of from about 0° C. to about 100° C., with aqueous ammonia in the absence or presence of an organic solvent. It is furthermore possible to synthesize aromatic sulfonamides, in accordance with methods that are described in the literature, from the acylated amines of formula XII by means of reaction with alkali metal-organic or alkaline earth metal-organic reagents, in an inert solvent under an inert gas atmosphere at temperatures of from about −100° C. to about 50° C., preferably of from about −100° C. to about 30° C., and with sulfur dioxide and subsequent thermal treatment with sulfamic acid.

If the group R² in the compound of formula XIII is a precursor of the final R² group, the conversion of the group R² can be effected either before or after introducing the sulfamoyl group SO₂NH₂. If it is effected after introducing the sulfamoyl group, it may be appropriate, when converting the R² group, to use a standard method to protect the sulfamoyl group reversibly, for example by converting it into the N,N-dimethylaminomethylenesulfamoyl group by reaction with a dimethylformamide acetal.

If the acyl group in the compound of formula XIII functions as a protecting group for the amino group, this protecting group can then be eliminated, after the sulfonamide group has been introduced, by treating with acids or bases. By treatment with aqueous acids or with acids in inert solvents the acid addition salt of the amino compound can be formed. Sulfuric acid, hydrohalic acids, such as hydrochloric acid or hydrobromic acid, phosphoric acids, such as orthophosphoric acid, or organic acids, for example, are suitable for carrying out this protecting group elimination. The elimination of the amino protecting group in the compound of formula XIII using bases can be effected in aqueous or inert solvents. Examples of suitable bases are alkali metal or alkaline earth metal hydroxides, such as sodium hydroxide, potassium hydroxide or calcium hydroxide, or alkali metal or alkaline earth metal alcoholates, such as sodium methoxide, sodium ethoxide, potassium methoxide or potassium ethoxide. The benzenesulfonamides of formula III can be prepared from the sulfonamide-substituted amines, or their acid addition salts, that have been prepared in this way, by acylation with substituted benzoic acids or benzoic acid derivatives, as explained above for the acylation of the compounds of formula XI.

The compounds of formula I can possess one or more chiral centers. When they are prepared, they can be obtained as racemates or, if optically active starting compounds are used, also in optically active form. If the compounds possess two or more chiral centers, they can then accrue, during the synthesis, as mixtures of racemates, and the individual compounds can be isolated in pure form, for example, by recrystallizing from inert solvents. If desired, racemates that have been obtained can be separated into their enantiomers using methods that are known per se. For example, diastereomers can be formed from the racemate by reaction with an optically active resolving agent. Examples of suitable resolving agents for basic compounds are optically active acids such as the R or the R,R or the S or the S,S form of tartaric acid, dibenzoyltartaric acid, diacetyltartaric acid, camphorsulfonic acids, mandelic acids, malic acid or lactic acid. The diastereomers can be separated in a manner known per se, for example by fractional crystallization, and the enantiomers can then be liberated from the diastereomers in a manner known per se. It is furthermore possible to effect a separation of the enantiomers by means of chromatography on optically active support materials.

Depending on the nature of the residues R¹, R², R³, E, X, Y and Z, in some cases a process from those described above for preparing the compounds of formula I will be unsuitable, or will it become necessary to take precautions for protecting active groups, for example. Such cases that occur relatively rarely, can be easily recognised by the skilled person, and no difficulty is involved in successfully using another of the above-described synthesis processes in such cases. Furthermore, with regard to the preparation of the compounds of formula I that are to be used according to the invention, reference is made to U.S. Pat. No. 5,574,069 (EP-A-612724) and U.S. Pat. No. 5,652,268 (EP-A-727416), whose content is incorporated herein by reference.

The suitability of a compound of formula I for treating pathological changes in blood pressure associated with septic shock or occurring in the generalized inflammatory syndrome (SIRS) state can be established, for example, in the pharmacological model in the pig that is described further below (endotoxin model, synonym: LPS model (LPS=lipopolysaccharide)). The effect can also be examined, for example, in rats, mice, cats, guinea pigs, rabbits, dogs or monkeys.

Due to the biological activity that has been found, a compound of formula I, or a physiologically tolerated salt thereof or solvate thereof, can be used in animals, preferably in mammals, and in particular in humans, as medicaments on their own, in mixtures with one another, for example as a mixture of two compounds of formula I and/or their physiologically tolerated salts, or together with other pharmacologically active compounds, in the treatment of septic shock or the generalized inflammatory syndrome (SIRS), in particular for treating pathological changes in blood pressure associated with septic shock or occurring in the generalized inflammatory syndrome (SIRS) state. Preferably a compound of formula I, or a physiologically tolerated salt thereof or solvate thereof, is used for this purpose in the form of pharmaceutical preparations (or pharmaceutical compositions). The present invention also relates to a method for treating septic shock or the generalized inflammatory syndrome (SIRS), in particular a method for treating pathological changes in blood pressure associated with septic shock or occurring in the generalized inflammatory syndrome (SIRS) state, in which method an effective dose of one or more compounds of formula I, or physiologically tolerated salts thereof or solvates thereof, is/are administered to a human or an animal. The invention furthermore relates to pharmaceutical preparations (or pharmaceutical compositions) for treating septic shock or the generalized inflammatory syndrome (SIRS), in particular pharmaceutical preparations for treating pathological changes in blood pressure associated with septic shock or occurring in the generalized inflammatory syndrome (SIRS) state, which preparations comprise an effective dose of one or more compounds of formula I, or physiologically tolerated salts thereof or solvates thereof, together with a pharmaceutically acceptable carrier, i.e., one or more pharmaceutically acceptable vehicles or carrier substances or auxiliary substances or additives.

Medicaments that are to be used according to the invention and that comprise a compound of formula I, or physiologically tolerated salt thereof or solvate thereof, can be administered enterally, for example orally or rectally, for example in the form of pills, tablets, film tablets, sugar-coated tablets, granules, hard gelatin capsules, soft gelatin capsules, suppositories, solutions, such as aqueous, alcoholic or oily solutions, juices, drops, syrups, emulsions or suspensions. The medicaments can also be administered parenterally, for example subcutaneously, intramuscularly or intravenously, in the form of injection solutions or infusion solutions. Other examples of suitable forms of administration are percutaneous or topical administration, for example in the form of ointments, creams, pastes, lotions, gels, sprays, powders, foams, aerosols or solutions, or use in the form of implants. In the use according to the present invention it is particularly suitable to use a compound of formula I, or physiologically tolerated salt thereof or solvate thereof, or the medicaments comprising them, by injection or infusion. Preferred forms of medicaments according to the invention thus include injection solutions and infusion solutions and pharmaceutical preparations from which injection solutions and infusion solutions are obtained, for example by a adding a liquid carrier substance.

The pharmaceutical preparations to be employed according to the invention can be produced using the known standard methods for producing pharmaceutical preparations. For this, one or more compounds of formula I, or physiologically tolerated salts thereof or solvates thereof, is/are mixed together with one or more solid or liquid galenic carrier substances and/or additives or auxiliary substances and, if a combination preparation is desired, additional pharmaceutically active ingredients having a therapeutic or prophylactic effect, and brought into a suitable administration form or dosage form that can then be used as a medicament in human medicine or veterinary medicine. The pharmaceutical preparations comprise a therapeutically or prophylactically effective dose of a compound of formula I, or physiologically tolerated salt thereof or solvate thereof, that normally amounts to from about 0.5 to about 90 per cent by weight of the pharmaceutical preparation. While the quantity of active compound of formula I, or physiologically tolerated salt thereof or solvate thereof, in the pharmaceutical preparations is normally from about 0.2 mg to about 1000 mg, preferably from about 1 mg to about 500 mg, per dose unit, it can also be higher depending on the nature of the pharmaceutical preparation.

Suitable carrier substances for producing pharmaceutical preparations are organic or inorganic substances that are suitable, for example, for enteral (for example oral) or parenteral (for example intravenous) administration or topical uses and which do not react with the active compounds in an undesirable manner, for example water, saline, vegetable oils, alcohols, such as ethanol, isopropanol or benzyl alcohols, 1,2-propanediol, polyethylene glycols, dimethylacetamide, glyceryl triacetate, gelatin, carbohydrates such as lactose or starch, talc, lanolin or vaseline. It is also possible to use mixtures of two or more carrier substances, for example mixtures of two or more solvents, in particular mixtures of one or more organic solvents with water. Additives or auxiliary substances which can be present in the pharmaceutical preparations include stabilizing agents, wetting agents, emulsifiers, solubilizers, thickeners, salts, for example for influencing the osmotic pressure, glidants, preservatives, dyes, flavorings, aromatizing substances and/or buffering substances, such as, for example stearic acid, magnesium stearate, polyvinylpyrrolidone, sodium chloride, silica, cellulose derivatives, etc. The pharmaceutical preparations can also comprise one or more additional active ingredients, for example vitamins or protein C activators. A compounds of formula I, or physiologically tolerated salt thereof or solvate thereof, can also be lyophilized and the resulting lyophilisates can, for example, be used for producing injection preparations and infusion preparations. Liposomal preparations are also suitable, for example for topical use.

The dose of the active compound of formula I, or physiologically tolerated salt thereof or solvate thereof, that is to be administered in the use according to the invention depends on the individual case and, as usual, has to be adapted to the individual circumstances in order to achieve an optimal effect. Thus, it depends on the circumstances of the specific case, on the sex, age, weight and individual responsiveness of the human or animal to be treated, on the strength and duration of effect of the compounds employed, on whether the therapy or prophylaxis is being conducted acutely or over a relatively long period of time, or on whether other active compounds, such as Xigris™, in addition to compounds of formula I, are being administered. In general, a dose range for treating septic shock, sepsis or generalized inflammatory syndrome (SIRS) in humans of from about 0.1 mg to about 100 mg per kg and day is appropriate for achieving the intended effect when the dose is being administered to an adult weighing about 75 kg. Preference is given to a dose range of from about 1 mg to about 30 mg per kg and day (in each case mg per kg of body weight). The daily dose can be administered as one single dose or be subdivided into several individual doses, for example one, two, three or four individual doses. The dose can, for example, be administered as a bolus or continuously, for example by means of infusion or continuous infusion. Where appropriate, it may be necessary to deviate upwards or downwards from the abovementioned daily dose depending on the individual response.

The foregoing may be better understood by reference to the following Examples, which are presented for illustration and not intended to limit the scope of the invention.

### EXAMPLES

### Example 1

### 1-(5-(2-(5-Chloro-2-methoxybenzamido)ethyl)-2-(2-methoxyethoxy)phenylsulfonyl)-3-methylthiourea

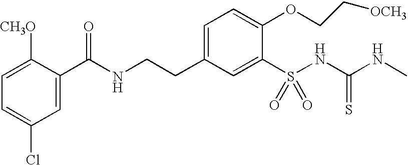

670 mg of 5-(2-(5-chloro-2-methoxybenzamido)ethyl)-2-(2-methoxyethoxy)-benzenesulfonamide were dissolved in 10 ml of absolute dimethylformamide and 70 mg of 60% sodium hydride were added. The mixture was stirred at room temperature for 20 min and 1.6 ml of a 1M solution of methyl isothiocyanate in dimethylformamide were then added dropwise. The mixture was heated at 80° C. for 1.5 h. After it had been cooled down, the mixture was added dropwise to 100 ml of 1N hydrochloric acid. The resulting mixture was then extracted with ethyl acetate, the organic phase was separated off and dried and the solvent removed in vacuo. The resulting solid was dissolved in a little hot ethanol and precipitated with water. Yield 720 mg. Melting point 134° C.

Preparation of 5-(2-(5-chloro-2-methoxybenzamido)ethyl)-2-(2-methoxyethoxy)-benzenesulfonamide

a) 4-(2-Trifluoroacetamidoethyl)-2-(N,N-dimethylaminomethyleneaminosulfonyl)-anisole

32.6 g (100 mmol) of 2,2,2-trifluoro-N-(2-(4-methoxy-3-sulfamoylphenyl)ethyl)-acetamide (obtainable from 2-(4-methoxyphenyl)ethylamine by conversion into the trifluoroacetamide, reaction with chlorosulfonic acid and reaction with ammonia) were dissolved in 70 ml of dimethylformamide and 16 ml (120 mmol) of N,N-dimethylformamide dimethyl acetal were added. The mixture was stirred for 3 h at room temperature and poured onto ice/NaHSO₄ solution (5%). The precipitate was filtered off with suction, washed with water and dried. 32.5 g (85%) of the title compound were obtained as a white solid. Melting point: 143-144° C. MS (ESI) m/e 382 (M+H⁺).

b) 4-(2-Trifluoroacetamidoethyl)-2-(N,N-dimethylaminomethyleneaminosulfonyl)-phenol hydrobromide

32.5 g (85 mmol) of the compound of step a) were dissolved in 450 ml of dichloromethane and 100 ml (100 mmol) of a 1M solution of boron tribromide in dichloromethane were added slowly. The mixture was stirred at room temperature for 5 h, treated with 150 ml of methanol and poured onto 2 l of diisopropyl ether. The precipitate was filtered off. Yield: 36.0 g (95%) of the title compound as a colorless solid. Melting point: 160-161° C. MS (ESI) m/e 368 (M+H⁺).

c) 2-(4-(2-Methoxyethoxy)-3-sulfamoylphenyl)ethylamine hydrochloride

2.7 g (6 mmol) of the compound of step b), 2.92 g (21 mmol) of 2-bromoethyl methyl ether and 2.1 g (15 mmol) of potassium carbonate were stirred in 100 ml of dimethylformamide for 3 h at 70° C. The mixture was then diluted with ethyl acetate, washed with aqueous sodium chloride solution, and the organic phase was dried and concentrated in vacuo. 1.9 g (85%) of the intermediate were obtained by chromatographing the residue with ethyl acetate. The intermediate was then heated under reflux for 8 h in a mixture of 25 ml of methanol and 25 ml of 5.5 N hydrochloric acid. The mixture was concentrated, the residue was washed with ethanol; and the precipitate was filtered off with suction and washed with ethanol. 1.2 g (83%) of the title compound were obtained as a colorless solid. Melting point: 195-197° C. MS (ESI) m/e 275 (M+H⁺).

d) 5-(2-(5-Chloro-2-methoxybenzamido)ethyl)-2-(2-methoxyethoxy)-benzenesulfonamide

0.75 g (3.65 mmol) of 5-chloro-2-methoxybenzoyl chloride were added to a solution of 1.1 g (3.5 mmol) of the compound of step c) and 1 ml of triethylamine in 20 ml of dry tetrahydrofuran and the reaction mixture was stirred at room temperature for 1.5 h. 80 ml of water were then added, and the precipitated product was filtered off, washed with water and dried in vacuo. Yield: 1.32 g (85%).

### Example 2

### 1-(5-(2-(5-Chloro-2-methoxybenzamido)ethyl)-2-methoxyphenylsulfonyl)-3-methylthiourea

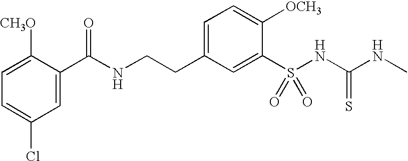

400 mg of 5-(2-(5-chloro-2-methoxybenzamido)ethyl)-2-methoxybenzenesulfonamide were dissolved in 5 ml of absolute dimethylformamide and 42 mg of 60% sodium hydride were added. The mixture was stirred at room temperature for 30 min. 1.2 ml of a 1M solution of methyl isothiocyanate in dimethylformamide were then added dropwise and the mixture was heated at 80° C. for 1.5 h. After it had been cooled down, the reaction mixture was added dropwise to 50 ml of 1N hydrochloric acid. The precipitated product was filtered off with suction and dried. Yield 96%. Melting point 190-193° C.

### Example 3

Aqueous Solution for Intravenous Administration

In order to prepare 10 ml of a solution for intravenous application which contains 10 mg of active compound per ml, 100 mg of the sodium salt of 1-(5-(2-(5-chloro-2-methoxy-benzamido)ethyl)-2-(2-methoxyethoxy)phenylsulfonyl)-3-methylthiourea were dissolved in 10 ml of isotonic (0.9%) sodium chloride solution.

Pharmacological Investigations

Anesthetized pigs were infused continuously with lipopolysaccharide (LPS) (0.15 μg/kg/h; n=7). This led to a decrease in the peripheral resistance. After 3.9 hours, 1-(5-(2-(5-chloro-2-methoxybenzamido)ethyl)-2-(2-methoxyethoxy)phenyl-sulfonyl)-3-methylthiourea sodium salt was administered at a dose of 5-10 mg/kg (intravenously; aqueous solution). As a result, the peripheral mean arterial blood pressure rose significantly by 19.6±3.2 mm Hg (p<0.001). The peripheral resistance, which under the effect of the endotoxin had fallen to 60.8±4.1% of the starting value that had been present prior to administering the endotoxin, rose to 80.8±5.1% of the starting value that had been present prior to administering the endotoxin (p<0.0001).

When the 1-(5-(2-(5-chloro-2-methoxybenzamido)ethyl)-2-(2-methoxyethoxy)-phenylsulfonyl)-3-methylthiourea sodium salt was administered as an infusion (total dose 5-10 mg/kg), a marked improvement was already seen after a dose of 1-2.5 mg/kg had been infused.

In another experimental approach performed on anesthetized pigs, 1 μg/kg of LPS was administered as a bolus (n=5). This led, within 15-20 min, to a dangerous increase in the systolic pulmonary arterial pressure from 30.6±0.7 mm Hg to 67.2±6.0 mm Hg. Administration of 5 mg/kg of 1-(5-(2-(5-chloro-2-methoxybenzamido)-ethyl)-2-(2-methoxyethoxy)phenylsulfonyl)-3-methylthiourea sodium salt (intravenously; bolus) lowered the systolic pulmonary arterial pressure significantly to 46.6±4.0 mm Hg (p<0.01).

These experimental data prove that in septic shock and in the generalized inflammatory syndrome (SIRS) state a compound of formula I raises the peripheral arterial blood pressure and at the same time lower the increased pulmonary arterial pressure, and demonstrate the superiority of the compounds of formula I, as compared with other vasoconstrictive substances, in the treatment of septic shock.

We claim:

1. A method of treating a patient suffering from septic shock or generalized inflammatory syndrome, comprising administering to the patient a pharmaceutically acceptable amount of a benzenesulfonyl(thiourea or urea) of formula I, 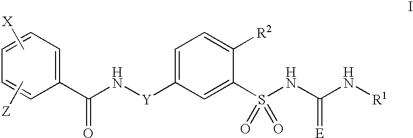
wherein
R¹ is hydrogen, (C₁-C₈)-alkyl-, (C₃-C₈)-cycloalkyl-, (C₃-C₈)-cycloalkyl-(C₁-C₄)-alkyl- or fluoro-(C₁-C₈)-alkyl-;
R² is (C₁-C₆)-alkoxy-, (C₃-C₈)-cycloalkyloxy-, (C₃-C₈)-cycloalkyl-(C₁-C₄)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;
E is oxygen or sulfur;
Y is a hydrocarbon residue of formula —(CR³₂)ₙ—, wherein the residues R³, all independently of each other, are hydrogen or (C₁-C₂)-alkyl-, and n is 1, 2, 3 or 4;
X is hydrogen, halogen or (C₁-C₆)-alkyl-; and
Z is halogen, (C₁-C₄)-alkyl, fluoro-(C₁-C₄)-alkyl-, (C₁-C₄)-alkoxy- or fluoro-(C₁-C₄)-alkoxy-, or
a physiologically tolerable salt thereof or solvate thereof.

2. The method as claimed in claim 1, wherein in formula I,
R¹ is hydrogen or (C₁-C₆)-alkyl-;
R² is (C₁-C₆)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-; and
Z is halogen, (C₁-C₄)-alkyl- or (C₁-C₄)-alkoxy-.

3. The method as claimed in claim 2, wherein in formula I,
R¹ is (C₁-C₄)-alkyl-;
R² is methoxy or 2-methoxy-ethoxy-;
Y is the hydrocarbon residue of formula —(CR³₂)ₙ—, wherein the residues R³ all are hydrogen, and n is 2;
X is chlorine, fluorine or (C₁-C₃)-alkyl-; and
Z is chlorine, fluorine, (C₁-C₃)-alkyl- or (C₁-C₃)-alkoxy-.

4. The method as claimed in claim 1, wherein the benzenesulfonyl(thiourea or urea) of formula I is 1-(5-(2-(5-chloro-2-methoxybenzamido)ethyl)-2-(2-methoxyethoxy)-phenylsulfonyl)-3-methylthiourea, or a physiologically tolerable salt thereof or solvate thereof.

5. The method as claimed in claim 1, wherein the benzenesulfonyl(thiourea or urea) of formula I is 1-(5-(2-(5-chloro-2-methoxybenzamido)ethyl)-2-methoxyphenyl-sulfonyl)-3-methylthiourea, or a physiologically tolerable salt thereof or solvate thereof.

6. The method as claimed in claim 1, wherein the administering is by injection or infusion.

7. The method as claimed in claim 1, wherein the benzenesulfonyl(thiourea or urea) of formula I is in the form of a sodium salt thereof.

8. A method of treating a patient suffering from pathological changes in blood pressure arising from septic shock or generalized inflammatory syndrome, comprising administering to the patient a pharmaceutically acceptable amount of a benzenesulfonyl(thiourea or urea) of formula I, 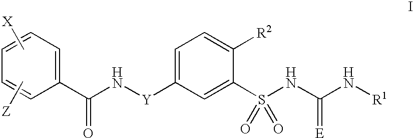
wherein
R¹ is hydrogen, (C₁-C₈)-alkyl-, (C₃-C₈)-cycloalkyl-, (C₃-C₈)-cycloalkyl-(C₁-C₄)-alkyl- or fluoro-(C₁-C₈)-alkyl-;
R² is (C₁-C₆)-alkoxy-, (C₃-C₈)-cycloalkyloxy-, (C₃-C₈)-cycloalkyl-(C₁-C₄)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;
E is oxygen or sulfur;
Y is a hydrocarbon residue of formula —(CR³₂)ₙ—, wherein the residues R³, all independently of each other, are hydrogen or (C₁-C₂)-alkyl-, and n is 1, 2, 3 or 4;
X is hydrogen, halogen or (C₁-C₆)-alkyl-; and
Z is halogen, (C₁-C₄)-alkyl, fluoro-(C₁-C₄)-alkyl-, (C₁-C₄)-alkoxy or fluoro-(C₁-C₄)-alkoxy-, or
a physiologically tolerable salt thereof or solvate thereof.

9. The method as claimed in claim 8, wherein in formula I,
R¹ is hydrogen or (C₁-C₆)-alkyl-;
R² is (C₁-C₆)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-; and
Z is halogen, (C₁-C₄)-alkyl- or (C₁-C₄)-alkoxy-.

10. The method as claimed in claim 9, wherein in formula I,
R¹ is (C₁-C₄)-alkyl-;
R² is methoxy or 2-methoxy-ethoxy-;
Y is the hydrocarbon residue of formula —(CR³₂)ₙ—, wherein the residues R³ all are hydrogen, and n is 2;
X is chlorine, fluorine or (C₁-C₃)-alkyl-; and
Z is chlorine, fluorine, (C₁-C₃)-alkyl- or (C₁-C₃)-alkoxy-.

11. The method as claimed in claim 8, wherein the benzenesulfonyl(thiourea or urea) of formula I is 1-(5-(2-(5-chloro-2-methoxybenzamido)ethyl)-2-(2-methoxyethoxy)-phenylsulfonyl)-3-methylthiourea, or a physiologically tolerable salt thereof or solvate thereof.

12. The method as claimed in claim 8, wherein the benzenesulfonyl(thiourea or urea) of formula I is 1-(5-(2-(5-chloro-2-methoxybenzamido)ethyl)-2-methoxyphenylsulfonyl)-3-methylthiourea, or a physiologically tolerable salt thereof or solvate thereof.

13. The method as claimed in claim 8, wherein the administering is by injection or infusion.

14. The method as claimed in claim 8, wherein the benzenesulfonyl(thiourea or urea) of formula I is in the form of a sodium salt thereof.

15. A method of treating a patient suffering from a decrease in peripheral (systemic) blood pressure and, at the same time, an increase in pulmonary arterial pressure, comprising administering to the patient a pharmaceutically acceptable amount of a benzenesulfonyl(thiourea or urea) of formula I, 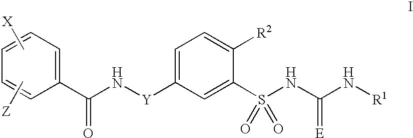
wherein
R¹ is hydrogen, (C₁-C₈)-alkyl-, (C₃-C₈)-cycloalkyl-, (C₃-C₈)-cycloalkyl-(C₁-C₄)-alkyl- or fluoro-(C₁-C₈)-alkyl-;
R² is (C₁-C₆)-alkoxy-, (C₃-C₈)-cycloalkyloxy-, (C₃-C₈)-cycloalkyl-(C₁-C₄)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-;
E is oxygen or sulfur;
Y is a hydrocarbon residue of formula —(CR³₂)ₙ—, wherein the residues R³, all independently of each other, are hydrogen or (C₁-C₂)-alkyl-, and n is 1, 2, 3 or 4;
X is hydrogen, halogen or (C₁-C₆)-alkyl-; and
Z is halogen, (C₁-C₄)-alkyl, fluoro-(C₁-C₄)-alkyl-, (C₁-C₄)-alkoxy or fluoro-(C₁-C₄)-alkoxy-, or
a physiologically tolerable salt thereof or solvate thereof.

16. The method as claimed in claim 15, wherein in formula I,
R¹ is hydrogen or (C₁-C₆)-alkyl-;
R² is (C₁-C₆)-alkoxy-, (C₁-C₆)-alkoxy-(C₁-C₄)-alkoxy- or (C₁-C₆)-alkoxy-(C₁-C₄)-alkoxy-(C₁-C₄)-alkoxy-; and
Z is halogen, (C₁-C₄)-alkyl- or (C₁-C₄)-alkoxy-.

17. The method as claimed in claim 15, wherein in formula I,
R¹ is (C₁-C₄)-alkyl-;
R² is methoxy or 2-methoxy-ethoxy-;
Y is the hydrocarbon residue of formula —(CR³₂)ₙ—, wherein the residues R³ all are hydrogen, and n is 2;
X is chlorine, fluorine or (C₁-C₃)-alkyl-; and
Z is chlorine, fluorine, (C₁-C₃)-alkyl- or (C₁-C₃)-alkoxy-.

18. The method as claimed in claim 15, wherein the benzenesulfonyl(thiourea or urea) of formula I is 1-(5-(2-(5-chloro-2-methoxybenzamido)ethyl)-2-(2-methoxyethoxy)-phenylsulfonyl)-3-methylthiourea, or a physiologically tolerable salt thereof or solvate thereof.

19. The method as claimed in claim 15, wherein the benzenesulfonyl(thiourea or urea) of formula I is 1-(5-(2-(5-chloro-2-methoxybenzamido)ethyl)-2-methoxyphenylsulfonyl)-3-methylthiourea, or a physiologically tolerable salt thereof or solvate thereof.

20. The method as claimed in claim 15, wherein the administering is by injection or infusion.

21. The method as claimed in claim 15, wherein the benzenesulfonyl(thiourea or urea) of formula I is in the form of a sodium salt thereof.
